(19) United States
(12) Patent Application Publication
(10) Pub. No.: US 2004/0002116 A1
(43) Pub. Date: Jan. 1, 2004

(54) Simple method of cloning, overexpressing and purifying lens protein tau-crystallin

(75) Inventors: Yogendra Sharma, Hyderabad (IN); Ramesh K. Aggarwal, Hyderabad (IN)

Correspondence Address: DRINKER BIDDLE & REATH, ONE LOGAN SQUARE, 18TH AND CHERRY STREETS, PHILADELPHIA, PA, 19103-6996, US

(73) Assignee: Council of Scientific and Industrial Research, New Delhi (IN)

(21) Appl. No.: 10/346,847

(22) Filed: Jan. 17, 2003

Related U.S. Application Data
(60) Provisional application No. 60/351,112, filed on Jan. 23, 2002.

(51) Int. Cl. G01N 33/53; C12P 21/02; C12N 1/21; C07K 14/47; C07H 21/04; C12P 19/34; C12N 15/74

(52) U.S. Cl. 435/7.1; 435/69.1; 435/91.2; 435/252.33; 435/320.1; 530/350; 536/23.5

(57) ABSTRACT

The present invention relates to a simple method of cloning, overexpressing, and purifying eye lens protein tau-crystallin, said method comprising steps of isolating total RNA from eye lens, synthesizing cDNA, cloning in an expression host, and purifying tau-crystallin.

### FIELD OF INVENTION

The present invention relates to a simple method of cloning, overexpressing, and purifying eye lens protein tau-crystallin, said method comprising steps of isolating total RNA from eye lens, synthesizing cDNA, cloning in an expression host, and purifying tau-crystallin.

### BACKGROUND AND PRIOR ART REFERENCES

Tau (-c)-crystallin is an important taxon-specific crystallin present in many reptilian and a few avian lenses (1). It is identical to a-enolase, a house-keeping enzyme of the glycolytic pathway indicating its recruitment for a dual role—as a structural protein in lens and as a metabolic enzyme in other tissues (2,3)-t-Crystallin was first identified and characterized from lamprey and turtles (4,5) and thus was named as τ (tau; derived from turtle) (5). Later, it was also found in duck lens and was shown to have sequence similarity to a-enolase (3,6).

Not much data are available on the structure and conformation of this crystallin barring a few reports from turtle and lamprey lenses. One of the reasons for this lacuna is the extreme difficulty in procuring the source lenses from such rare animals owing to various national and international wild life conservation acts. To overcome these constraints, it would be prudent if these crystallins were prepared by bacterial overexpression. We have been interested in ?—crystallin from crocodilian eye lens and have cloned and sequenced complete cDNA of this crystallin¹. It is the minor crystallin in the crocodile lens, comprising just about 3-4% of the total lens proteins, thus limiting the biochemical and biophysical studies on this crystallin.

To overcome this problem, we have developed a method for overexpression and purification of crocodile t-crystallin in bacterial expression host E. coli. Moreover, we establish that the recombinant protein is a x-crystallin (monomer) and not alpa-enolase (dimer).

### OBJECT OF THE PRESENT INVENTION

The main object of the present invention is to develop a clone of the gene coding for protein Tau-crystallin.

Another object of the present invention is to develop a method to overexpress the gene coding for protein Tau-crystallin.

Yet another object of the present invention is to obtain protein Tau-crystallin.

Still another object of the present invention is to isolate protein Tau-crystallin of high purity.

Still another object of the present invention to develop a method to isolate protein Tau-crystallin in a short span of time.

Still another object of the present invention is to characterize the protein Tau-crystallin.

### SUMMARY OF THE PRESENT INVENTION

The present invention relates to a simple method of cloning, overexpressing, and purifying eye lens protein tau-crystallin, said method comprising steps of isolating total RNA from eye lens, synthesizing cDNA, cloning in an expression host, and purifying tau-crystallin.

### DETAILED DESCRIPTION OF THE PRESENT INVENTION

Accordingly, the present invention relates to a simple method of cloning, overexpressing, and purifying eye lens protein tau-crystallin, said method comprising steps of isolating total RNA from eye lens, synthesizing cDNA, cloning in an expression host, and purifying tau-crystallin.

A simple method of cloning, overexpressing, and purifying eye lens protein tau-crystallin, said method comprising steps of: isolating total RNA from eye lens, synthesizing cDNA from the total RNA using a mixture of random primers and oligo dT primer in RT-PCR, designing both forward and reverse primers using synthesized cDNA to obtain PCR product, cloning the said PCR product as an insert in a plasmid, releasing the insert by double-digesting it with restriction enzymes, sub-cloning the released insert in an expression vector, transforming the sub-clone into an E.Coli expression host, inducing the host with IPTG, pelleting the induced host, re-suspending the pellet in lysis buffer for braking the cells, spinning the broken cells to obtain supernatant, loading the supernatant for gel filtration chromatography to obtain fractions, pooling the fractions that contain tau crystallin, purifying tau-crystallin from pooled fractions by ion-exchange Mono-Q FPLC, determining molecular weight and quaternary structure of the purified tau-crystallin using gel filtration superose 12 FPLC, determining the purity of tau-crystallin by SDS-polyacrylamide gel electrophoresis, determining secondary and tertiary structure by Far and Near-UV spectra respectively, obtaining cloned, overexpressed, and purified eye lens protein tau-crystallin.

In an embodiment of the present invention, wherein obtaining RNA from embryonic lens. n another embodiment of the present invention, wherein synthesizing cDNA using superscript II RNaseH Reverse Transcriptase Kit.

In yet another embodiment of the present invention, wherein forward primer shows sequence of SEQ ID No. 1.

In still another embodiment of the present invention, wherein the reverse primer shows sequence of SEQ ID No. 2.

In still another embodiment of the present invention, wherein the plasmid is bluescript pBSKSII.

In still another embodiment of the present invention, A method as claimed in claim 1, wherein the restriction enzymes are selected from a group comprising NdeI and XhoI.

In still another embodiment of the present invention, wherein the expression host is E. Coli BL21 (DE3).

In still another embodiment of the present invention, wherein IPTG is at concentration of about 1 mM.

In still another embodiment of the present invention, wherein SDS-polyacrylamide gel electrophoresis is on a discontinuous buffer system on about 10% polyacrylamide.

In still another embodiment of the present invention, wherein protein tau crystallin constitute about 80% of the total protein expressed in transformed expression host.

In still another embodiment of the present invention, wherein the overexpressed protein tau crystallin maintains native confirmation in expression host.

In still another embodiment of the present invention, wherein the purity of the protein is above 90%.

Accordingly the Present invention relates to a gene clone expressing protein tau-crystallin and a method of overexpressing said clone and a process of producing said protein in extremely pure state from said clone by using simplified purification methodology comprising two step chromatography technique.

T-Crystallin is a taxon-restricted crystallin found in eye lenses of reptiles and a few avian species but presumably absent in mammals. The level of T-crystallin in the lens varies among different species. In the crocodile lens, it is the least abundant crystallin and is present in trace amount. We present a method for cloning, overexpression and purification of crocodilian t-crystallin utilizing a combination of gel filtration and ion-exchange chromatography yielding an extremely purified protein. The protein gets profusely expressed resulting in a fairly high yield and exists as a monomeric entity of 47.5 kDa molecular mass. The recombinant T-crystallin exists in a properly folded native state as probed by circular dichroism and fluorescence spectroscopy.

RNA Isolation

Total RNA was extracted from the embryonic lens (developmental stages 21-25) of Indian mugger (Crocodylus palustris) using Trizol (Gibco BRL). cDNA was synthesized using Superscript II RNaseH Reverse Transcriptase kit (Life Technologies) using a mixture of random primer and oligo dT primer.

Primer Design and RT-PCR

Primers for i-crystallin were designed from the sequence of t-crystallin cDNA¹. The forwardprimer was selected so as to have an NdeI site at the initiating codon ATG. The reverse primer was selected from the 3′-UTR region just downstream of the stop codon and was engineered to introduce a XhoI site. The sequences of both primers were: Forward of SEQ ID No. 1: 5′-CAACATATGTCAGTTCTCAAGG-3′ Reverse of SEQ ID No. 2: 5′-GGCAGCTGCTGTTCTCGAGATAA-3′

PCR was performed on a MJ Research Thermal Cycler in a total volume of 50 jil. The conditions for PCR were: denaturation at 94° C. for 1 min, annealing at 45° C. for 1.5 min and extension at 72° C. for 2 min for 30 cycles. The PCR product (1320 bp) was checked on a 1.5% agarose gel, eluted using Qiagen gel extraction kit and verified by DNA sequencing using the same set of primers. The sequence (1320 bp) was analyzed by BLAST software program of NCBI(7).

Sub-Cloning in pET21a

The PCR product was blunt-end cloned in a plasmid bluescript pBSKSII and the insert was released by double digestion using NdeI and XhoI restriction enzymes (New England BioLab). This insert with overhangs was sub-cloned in a pET21a expression vector (Novagen) in NdeI-XhoI site, which provided the first codon ATG at the NdeI site. This clone was transformed into BL21(DE3) expression host.

T-Crystallin Expression in E. coli

The transformed BL21(DE3) E. Coli cells were grown in LB medium up to A6oo⁼0.6 before being induced with IPTG at 1 mM final concentration. The growth was allowed for 3 hours post-induction and ACOO was recorded before harvesting the cells. Equal amount of proteins before and after induction were checked on 10% SDS-PAGE followed by Coomassie brilliant blue or silver staining as the case may be.

Gel Filtration Chromatography

Cells from 50 ml of induced culture were pelleted down and re-suspended in 3 ml of lysis buffer (50 mM Tris, pH 8.0, 100 mM Nad, 1 mM EDTA, 1 mM DTT and 1 mM PMSF). 100 ul of 10 mg/ml Lysozyme was added and the mixture was incubated on ice/or 30 minutes followed by sonication to complete the cell breakage. The sample was spun at 15K/30V4° C., pellet removed and supernatant loaded onto a Bio-Gel A-1.5 m column (2 cm×80 cm). Fractions (3 ml) from the Bio-Gel A-1.5 m column were checked on 10% SDS-PAGE and those having i-crystallin were pooled together.

Mono-Q FPLC

Pooled fractions from Bio-Gel A-1.5 m having over-expressed t-crystallin were purified on Mono-Q HR 5/5 FPLC columns. The buffer employed was 50 mM Tris, pH 8.0, 1 mM EDTA, 1 mM DTT and the gradient was developed from 0 to 1 M NaCl.

Superose 12 FPLC

Superose 12 column was equilibrated with 50 mM Tris, pH 8, containing 1 mM DTT, 1 mM EDTA. The purified protein was loaded on to the column at a flow rate of 0.25 ml/min. For calculating the molecular weight, the column was calibrated with gel filtration standards (low molecular mass, Pharmacia Biotech) and the size of the protein was estimated based on the elution volume.

SDS-Polyacrylamide Gel Electrophoresis

SDS-polyacrylamide gel electrophoresis of proteins was performed on a discontinuous buffer system on 10% polyacrylamide.,Protein bands were visualized either by Coomassie brilliant blue or silver staining and photographed.

Circular Dichroism

Near- and far-UV CD spectra were recorded in a Jasco J-715 spectropolarimeter in a 1.0 and 0.05 cm path length cell in 50 mM Tris, pH 8.0, containing 100 mM NaCl, 1 mM DTT and 1 mM EDTA. Ellipticity values were expressed in millidegrees.

Steady-State Fluorescence

Emission spectrum was recorded using an excitation wavelength of 280 nm in a correct spectrum mode on a Hitachi F-4010 spectrofluorimeter. The buffer used was 50 mM Tris, pH 8, 100 mM NaCl, 1 mM DTT and 1 mM EDTA. The excitation and emission bandpasses were set at 5 nm.

### BRIEF DESCRIPTION OF THE ACCOMPANYING DRAWINGS

FIG. 1 shows (a) PCR product with the complete coding sequence of 1320 bp. Lane 1: 1 kb DNA ladder, lane 2: PCR product for T-crystallin cDNA. (b) 10% SDS-PAGE showing overexpression of i-crystallin. Lane 1: standard molecular weight markers, lane 2: control, uninduced cell lysate; lane 3: IPTG induced cell lysate overexpressing T-crystallin.

FIG. 2 shows (a) Bio-Gel A-1.5 m chromatography of the whole cell lysate in 50 ruM Tris, 100 mM NaCl, 1 mM DTT, 1 mM EDTA. The whole cell lysate was loaded and fractions were monitored at 280 nm. (b) SDS-PAGE analysis of peak fractions from Bio-Gel A-1.5 m column.

FIG. 3 shows SDS-PAGE-silver staining analysis of the Mono-Q flow through. M: Molecular weight markers; P: Mono-Q flow through having purified t.

FIG. 4 shows Superose 12 FPLC profile showing the elution of recombinant T-crystallin. The chromatography was performed in 50 mM Tris, pH 8 containing 100 mM NaCl, 1 mM DTT, 1 mM EDTA. The flow rate was 0.25 ml/min. The column was calibrated with molecular weight standards.

FIG. 5 shows (a) Far-UV and (b) near-UV CD of T-crystallin in 50 mM Tris, pH 8, 100 mM NaCl buffer containing 1 mM DTT and 1 mM EDTA. Protein concentration was 0.44 mg/ml. Cells of 0.05 cm and 1 cm path lengths were used for far- and near-UV CD respectively. Ellipticity values are represented in millidegrees.

FIG. 6 shows Steady-state fluorescence of T-crystallin. Protein solution (0.44 mg/ml) in 50 mM Tris, pH 8, containing 100 mM NaCl, 1 mM DTT and 1 mM EDTA was subjected to excitation at 280 nm and the emission spectrum was recorded in the correct spectrum mode. Excitation and emission bandpasses were set at 5 nm.

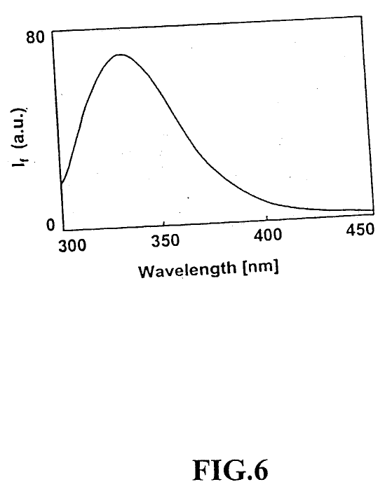

### RESULTS AND DISCUSSION

cDNA Subcloning and Protein Expression

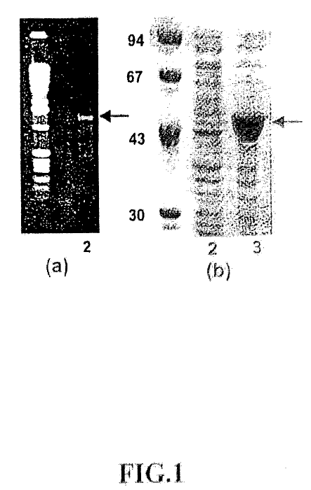

For cloning and over-expressing the t-crystallin gene, we performed PCR over the cDNA synthesized from the total RNA from crocodile embryonic lens. FIG. 1a shows the PCR product of the complete t-crystallin coding region (1320 bp). The product obtained pertains to the complete gene from the start to the stop codon with NdeI-XhoI site at the 5′ and 3′—ends respectively as confirmed by DNA sequencing. This blunt end product was cloned into the plasmid bluescript pBSKSII and the resulting recombinant plasmid was subjected to NdeI-XhoI double digestion to release the insert with the corresponding restriction overhangs. This insert with overhangs was sub-cloned into the NdeI-XhoI linearized pET21a expression vector to generate the T-crystallin expression clone (pET21a-t).

The pET21a-t clone was transformed into BL21(DE3) host and the protein expression was induced by IPTG as described in Materials and Methods. FIG. 1b shows the expression of this protein in transformed BL21(DE3) host E. coli cells both under control uninduced condition as well as after IPTG induction that resulted in profuse expression of T-crystallin to the extent of almost −80% of the total cellular proteins. The overexpressed protein confirms the expected molecular mass of 47.5 kDa as calculated based on the sequence analysis. Following expression, the protein was found to remain in the soluble fraction and did not form inclusion bodies.

Chromatographic Purification

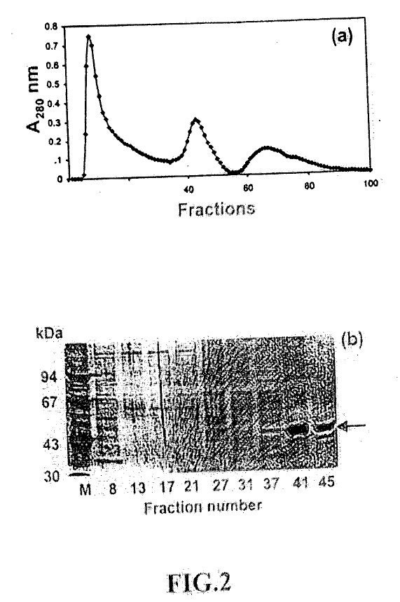

We then performed the purification of the over-expressed protein. After cell lysis, the sample was spun to get rid of pellet and the supernatant was subjected to gel filtration on Bio-Gel A-1.5 m column. FIG. 2a shows the Bio-Gel A-1.5 m chromatogram of the total cell lysate, which got fractionated as three well resolved peaks. SDS-PAGE analysis (FIG. 2b) of the peak fractions shows that T-crystallin elutes in the middle peak. The protein appeared homogeneous to the extent of −90% on SDS-PAGE after silver staining.

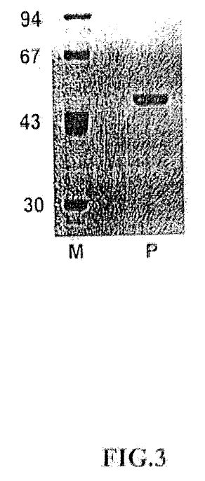

Though the T-crystallin could be purified almost completely on Bio-Gel A-1.5 m chromatography, the complete purification of the protein was achieved by ion-exchange FPLC using Mono-Q column (data not shown). In this case, the sole contaminating band (FIG. 2b) was retained on the column, whereas the T-crystallin eluted unbound as an uncontaminated, singular species as determined by SDS-PAGE-silver staining analysis (FIG. 3). Beginning with 100 ml of bacterial culture, the yield of the purified T-crystallin was about 5-6 mg.

Quaternary Structure

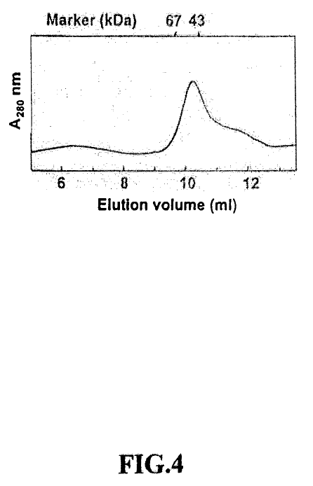

The molecular weight of the protein under denatured condition on SDS-PAGE shows the expected molecular mass of around 48 kDa. We also determined the molecular weight and quaternary structure by gel filtration on Superose 12 column. t-Crystallin is known to be identical to a-enolase in amino acid sequence (3). a-Enolase is known to exist as a dimmer whereas T-crystallin from eye lens is monomer. In order to see if the bacterially expressed protein is a monomer or a dimer, we performed gel filtration under native conditions. The protein, under the conditions employed, eluted as a monomer (FIG. 4). We further attempted to induce dimerization in the protein using magnesium ions, as they are implicated in a-enolase activity, and possibly in dimerization. Recombinant T-crystallin was found to remain monomer in the presence of 8 mM Mg²⁺ (data not shown), suggesting that the parameters deciding the monomeric/dimeric fate could be complicated.

Secondary and Tertiary Structure

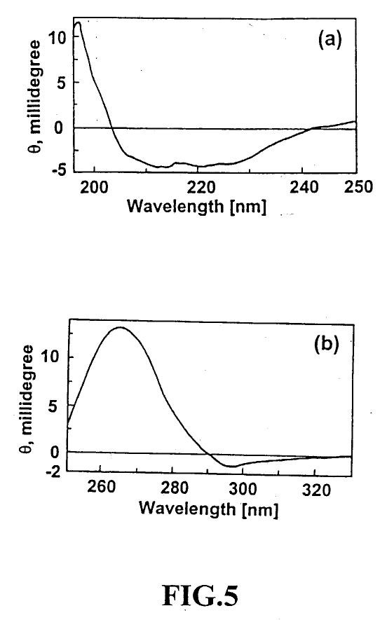

We determined the secondary structure of the recombinant T-crystallin by far-UV CD. The two minima at 222 and 208 nm indicate that the structure of the protein is dominated largely by alpha-helix (FIG. 5a). The CD profile is in good agreement with the only published report of the secondary structure of T-crystallin from turtle lens and matches closely in appearance (5). This suggests that the bacterially expressed T-crystallin is folded to its native conformation, making it suitable for structural and functional studies.

Near-UV CD of T-crystallin was performed to probe its tertiary structure, which is shown in FIG. 5b. There is a minimum at about 296 nm for Trp, followed by a very broad and dominant positive peak between 260-280 nm for aromatic amino acids. This composite peak is suggestive of the presence of a good tertiary structure. There is no data available on the tertiary structure of T-crystallin from crocodile lenses, or even closely related species such as alligator. The tertiary structure of the overexpressed protein does not match with the T-crystallin of turtle. This, however, is expected since there are species-specific variations in the protein sequences'. Far-and near-UV CD spectra in combination present T-crystallin with its own characteristic signature profile.

Trp Microenvironment

Steady-state protein fluorescence is a powerful tool to probe the folding state of the molecule by assessing the microenvironment of Trp, which also provides an insight into its tertiary structure. We recorded emission spectra of T-crystallin with the excitation wavelength set at 280 nm. The emission maximum is seen at around 330 nm, which indicates that Trp is buried in the hydrophobic environment. The emission maximum of crocodilian T-crystallin is close to that of turtle T-crystallin, which is 328 nm (5). Taken together, the far- and near-UV CD spectra and the fluorescence spectra strongly suggest that the recombinant protein is properly folded and is in a native conformation.

In summary, we present a novel and simple method for the over-expression and purification of a properly folded T-crystallin. Earlier purification of T-crystallin from turtle lenses involved a multi-step procedure consisting of isoelectric focusing, gel filtration and ion-exchange chromatography (5). The advantages of this method are the use of a simplified chromatographic procedure involving only two steps, which results in a significantly high yield of the protein with extreme purity. This purification procedure can be applied even on large scales making it a method of choice. The availability of purified i-crystallin will facilitate the detailed biochemical and structural studies of this important lens constituent, which would otherwise need to be isolated from the rare and endangered species such as crocodiles, where it is the least abundant crystallin. We further suggest that the bacterially expressed protein is a lens crystallin (monomer), and not a-enolase (dimer). The parameters dictating the monomer-dimer transition of this protein, which makes it exist either as a lens constituent, t-crystallin, or a metabolic enzyme, a-enolase are under investigation.

### REFERENCES

1. Wistow, G. J., and Piatigorsky, J. (1988) Lens crystaUins: the evolution and expression of proteins for a highly specialized tissue. Annu. Rev. Biochem. 57, 479-504.

2. Wistow, G., and Piatigorsky, J. (1987) Recruitment of enzymes as lens structural proteins. Science 236, 1554-1556.

3. Wistow, G. J., Lietman, T., Williams, L. A., Stapel, S. O., dejong, W. W., Horwitz, J., and Piatigorsky, J. (1988) Tau-crystallin/alpha-enolase: one gene encodes both an enzyme and a lens structural protein. J. Cell Biol. 107, 2729-2736.

4. Staple, S. O., and deJong, W. W. (1983) Lamprey 48 kDa lens protein represents a novel class of crystallins. FEBS Letters 162, 305-309.

5. Williams, L. A., Ding, L., Horwitz, J., and Piatigorsky, J. (1985) tau-Crystallin from the turtle lens: purification and partial characterization. Exp. Eye Res. 40, 741-749.

6. Kim, R. Y., Lietman, T., Piatigorsky, J., and Wistow, G. J. (1991) Structure and expression of the duck alpha-enolase/tau-crystallin-encoding gene. Gene 103, 193-200.

7. Altschul, S. F., Gish, W., Miller, W., Myers, E. W., and Lipman, D. J. (1990) Basic local alignment search tool. J. Mol. Biol. 215, 403-410.

1. A simple method of cloning, overexpressing, and purifying eye lens protein tau-crystallin, said method comprising steps of:
a. isolating total RNA from eye lens,
b. synthesizing cDNA from the total RNA using a mixture of random primers and oligo dT primer in RT-PCR,
c. designing both forward and reverse primers using synthesized cDNA to obtain PCR product,
d. cloning the said PCR product as an insert in a plasmid,
e. releasing the insert by double- digesting it with restriction enzymes,
f. sub-cloning the released insert in an expression vector,
g. transforming the sub-clone into an E.Coli expression host,
h. inducing the host with IPTG,
i. pelleting the induced host,
j. re-suspending the pellet in lysis buffer for braking the cells,
k. spinning the broken cells to obtain supernatant,
l. loading the supernatant for gel filtration chromatography to obtain fractions,
m. pooling the fractions that contain tau crystallin,
n. purifying tau-crystallin from pooled fractions by ion-exchange Mono-Q FPLC,
o. determining molecular weight and quaternary structure of the purified tau-crystallin using gel filtration superose 12 FPLC,
p. determining the purity of tau-crystallin by SDS-polyacrylamide gel electrophoresis,
q. determining secondary and tertiary structure by Far and Near-UV spectra respectively,
r. obtaining cloned, overexpressed, and purified eye lens protein tau-crystallin.

2. A method as claimed in claim 1, wherein obtaining RNA from embryonic lens.

3. A method as claimed in claim 1, wherein synthesizing cDNA using superscript II RNaseH Reverse Transcriptase Kit.

4. A method as claimed in claim 1, wherein forward primer shows sequence of SEQ ID No. 1.

5. A method as claimed in claim 1, wherein the reverse primer shows sequence of SEQ ID No. 2.

6. A method as claimed in claim 1, wherein the plasmid is bluescript pBSKSII.

7. A method as claimed in claim 1, wherein the restriction enzymes are selected from a group comprising NdeI and XhoI.

8. A method as claimed in claim 1, wherein the expression host is E. Coli BL21 (DE3).

9. A method as claimed in claim 1, wherein IPTG is at concentration of about 1 mM.

10. A method as claimed in claim 1, wherein SDS-polyacrylamide gel electrophoresis is on a discontinuous buffer system on about 10% polyacrylamide.

11. A method as claimed in claim 1, wherein protein tau crystallin constitute about 80% of the total protein expressed in transformed expression host.

12. A method as claimed in claim 1, wherein the overexpressed protein tau crystallin maintains native confirmation in expression host.

13. A method as claimed in claim 1, wherein the purity of the protein is above 90%.
